(19) United States
(12) Patent Application Publication
(10) Pub. No.: US 2015/0071890 A1
(43) Pub. Date: Mar. 12, 2015

(54) Compositions Containing Probiotics and a Beepollen/Clay Complex, Preparation Method Thereof and Uses of Same in Nutrition and Therapeutics

(71) Applicant: Laboratoire Beepratte, Paris (FR)

(72) Inventor: Alexandra Fregonese, Moncaut (FR)

(21) Appl. No.: 14/391,541

(22) PCT Filed: Apr. 10, 2013
(86) PCT No.: PCT/EP2013/057496

§ 371 Date: Oct. 9, 2014

(30) Foreign Application Priority Data
Apr. 10, 2012 (FR) 12 53253

(51) Int. Cl. A61K 47/46; A23L 1/30; A61K 35/74; A61K 9/48; A61K 9/19

(52) U.S. Cl. CPC A61K 47/46; A61K 9/48; A61K 9/19; A61K 35/745; A61K 35/747; A23L 1/3014
USPC 424/93.45; 424/93.1; 424/93.4

(57) ABSTRACT

The present invention relates to the use of a beepollen/clay complex as a support for administration of probiotics, as well as to compositions comprising one or several probiotics and said beepollen/clay complex.

The present invention relates to the administration of micro-organisms (bacteria or yeasts) with a probiotic purpose by means of a support based on beepollen and clay, as well as to compositions comprising said micro-organisms and said beepollen/clay complex.

According to the present invention, the term of micro-organism refers to micro-organisms (bacteria or yeasts) with a probiotic purpose.

Probiotics are micro-organisms such as non-pathogenic and non-toxic bacteria or yeasts, which, delivered alive in a sufficient amount in the intestine, contribute to maintaining equilibrium of the flora and to reinforcement of the intestinal mucosa. They are presently highly sought for beneficial effects which they would provide in term of nutrition and health.

Thus, the consultation of experts in October 2001 of the Food and Agriculture Organization (FAO)/WHO on the evaluation of health and nutritional properties of probiotics define probiotics as <<living micro-organisms which, when they are administered in a sufficient amount, give a health benefit to those who consume them>> and reported that probiotics may play an important role in immunological, digestive and respiratory functions and may have a significant effect for treating infectious diseases, notably in children.

Commercially available micro-organisms with a probiotic purpose, are generally freeze-dried and packaged in different forms (gelatin capsules, sachets, etc). Nevertheless, industrialization, gastric acidity and putting them in contact with biliary and pancreatic juices may be responsible for the loss of a large majority of these micro-organisms, which moreover have to be reactivated if they are freeze-dried. In the literature, several studies were dedicated to the viability of probiotics in an acid medium. Thus, the deleterious effect of acidity on the survival of certain probiotic strains (Bifido bacterium and Lactobacillus) were notably reported by Sun and al. International Journal of Food Microbiology, 61, 17-25 (2000), Sultana and al. International Journal of Food Microbiology, 62, 47-55 (2000), Favaro-Trindade and al. Journal of Microencapsulation, 19(4), 485-494 (2002) and Hansen and al. Food Microbiology, 19, 35-45 (2002).

Even among lactobacilli used by the dairy industry for making fermented milk and yoghurts, a large number does not survive in the acid environment of the stomach and therefore cannot be entitled to the definition of probiotics. Indeed, in order to be described as a “probiotic”, a strain of micro-organisms should arrive alive in a sufficient amount at the intestine and be established in the intestinal microflora.

Taking into account the benefits of probiotics, it is therefore desirable to make available formulations allowing the micro-organisms to arrive alive and in a sufficient amount in the intestine of the host.

Thus, it is essential to provide compositions which guarantee the probiotic effect of the micro-organisms added to the beepollen, by ensuring their bioavailability and their biological activity.

International application PCT/EP2011/072756 describes combinations of beepollen and of clay, here designated as a beepollen/clay complex, allowing stabilization, formulation as doses and administration of fresh beepollen, does not by any means contemplate its use as a support for administering micro-organisms with probiotic purpose characterized and cultivated beforehand, notably freeze-dried.

It has now been discovered and this is one of the objects of the present invention that said combinations of clay and of beepollen form an ideal administration system for micro-organisms with a probiotic purpose, by giving them the required protection against stomach acidity, biliary and pancreatic juices; by providing the food indispensable for their development and prior to their reactivation in the case of freeze-dried micro-organisms, by allowing them to give rise intra corpus to micro-organisms with great vitality, naturally adapted to the digestive environment; by delivering them alive to the intestine, by binding them therein and by releasing them therein.

According to the first object, the present invention, therefore relates to a composition comprising:
one or several micro-organisms with a probiotic purpose, and
a complex based on beepollen and clay.

According to another object, the present invention also relates to the use of a complex based on beepollen/clay as a support for administering micro-organisms with a probiotic purpose, notably freeze-dried, in particular for improving the bioload and the bioavailability of said micro-organisms.

Thus, according to the invention, it was demonstrated that the beepollen/clay complex forms an ideal protective and nutritional medium for micro-organisms with a probiotic purpose capable of ensuring their protection upon gastric passage, upon contact with biliary and pancreatic juices, providing the food required for their development and, prior to their reactivation in the case of freeze-dried micro-organisms.

With the complex it is possible to maintain the micro-organisms alive as far as the intestine and to preserve their biological characteristics, and therefore their physiological benefits on the equilibrium of the intestinal flora of the host.

In particular, the beepollen/clay complex provides an environment favorable for the multiplication intra corpus of living micro-organisms with a probiotic purpose, of great vitality, adapted to the digestive environment. Further, while the survival of micro-organisms with a probiotic purpose becomes a problem below a pH of 3, it was demonstrated that the beepollen/clay support of the compositions according to the invention gives the possibility of protecting the micro-organisms from gastric acidity, by maintaining the pH at a level compatible with cell life by a buffer effect which thus isolates the micro-organisms from the stomach pH and which allows birth intra corpus of micro-organisms at the end of the gastric passage. It was also demonstrated that this protection is continued upon passing into the intestine.

By birth intra corpus is understood the capability of the micro-organisms of multiplying in the beepollen/clay complex and of adapting to the physiological environment (notably the conditions of the digestive system) of the host to which they are administered.

By <<bee pollen/clay complex>> is meant any stable or <<anhydrous>> material resulting from the binding of beepollen from any plant species onto a silicate lamellar structure. International application PCT/EP2011/072756 described combinations of beepollen and of clay, and its contents is incorporated by reference herein.

In the sense of the invention, by <<beepollen>> is meant pollen taken by bees. This is the plant pollinator element extracted by the bee from the male organ of the flower, agglomerated from flower to flower and transported as far as the hive. It may be taken by any means, and notably just before entering the hive (by means of a pollen hatch), according to usual methods.

In the sense of the invention, this term of <<beepollen>> applies to pollen harvested by bees regardless of its extraction method and/or preservation.

In the sense of the invention, the term of <<beepollen>> designates a beepollen in any form with which it is possible to maintain the characteristics of fresh pollen. It therefore notably comprises fresh beepollen as well as beepollen frozen beforehand.

Within the scope of the present invention, the beepollen may originate from various plant species and is not limited to a particular flower. Mention may thus be made of beepollen from rye, maize, sunflower, rockrose, chestnut, kiwi, willow, poppy, lavender, and heather, etc.

The beepollen, notably frozen beepollen, may be commercially available for example at Pollenenergie.

The beepollen may also be of a variable composition, natively depending on the nature of the original plants as well as on the harvesting period, on the harvesting method or further on the geographical area (country, regions).

Thus, fresh beepollen suitable for the invention generally contains from 5% to 36% of water and from 64% to 95% of dry materials, among the latter:
2.5% to 3.8% of mineral salts;
4.2% to 19.8% of lipids;
8% to 30% of albumin;
5% to 7% of starch;
vitamins;
growth factors;
folic acid;
polyphenols;
phytosterols;
ferments and yeasts.

By <<clay>> is meant a material comprising clay minerals such as silicate hydrates, notably aluminum or magnesium silicates. Phyllosilicates having a laminated structure consisting of one or several sheets are notably preferred.

According to the Jozja classification, based on the thickness of the structure of the sheet, four groups of clays are distinguished:
i) 7 Å minerals: its thickness is of about 7 Å, this is the category of kaolinites (kaolin, halloysite . . . )
ii) 10 Å minerals: its thickness is of about 10 Å, this is the category of illites (illite, glauconite . . . )
iii) 14 Å minerals: its thickness is of about 14 Å, this is the category of smectites (montmorillonite, bentonite, ghassoul . . . )
iv) Fiber minerals: the thickness of the sheet is variable; they are called chlorites or fiber minerals (sepiolite, attapulgite . . . ).
   The term of <<clay>> according to the invention therefore refers to these four types of clay. As clayey minerals, mention may be made of illite, chlorite; glauconite, kaolinite, attapulgite, sepiolite and montmorillonite. As a clay, kaolin, illite, montmorillonite, attapulgite are notably preferred, and more particularly montmorillonite.

In the sense of the invention, by micro-organism is meant a microscopic organism notably including bacteria and yeasts, notably non-pathogenic and non-toxic.

In the sense of the invention, by micro-organism with a probiotic purpose are meant micro-organisms which are characterized and cultivated before their formulation, which, when they are delivered alive in a sufficient amount to the intestine, would give a health and/or nutrition benefit to the host.

The term of <<characterized>> used here refers here to a micro-organism for which the strain(s) which make it up, have(has) been identified, notably so as to know the nature of the strain(s) and optionally their concentration(s).

According to an embodiment, the micro-organisms characterized according to the invention are added to the beepollen. According to an embodiment, the micro-organisms added to the beepollen are distinct, notably by their nature, number, structure, from the micro-organisms naturally present in beepollen. According to an embodiment, freeze-dried micro-organisms are added.

As a yeast, mention may be made of a Saccharomyces cerivisiae. As bacteria, mention may essentially be made of lactic bacteria, such as lactobacilli, bifidobacteria and other lactic bacteria.

According to the invention:
as lactobacilli, mention may notably be made of Lactobacillus acidophilus, Lactobacillus amylovirus, Lactobacillus brevis, Lactobacillus casei, Lactobacillus cellobius,

Lactobacillus crispatus, Lactobacillus curvatus, Lactobacillus delbrueckii, Lactobacillus farciminis, Lactobacillus fermentum, Lactobacillus gasseri, Lactobacillus gallinarum, Lactobacillus helveticus, Lactobacillus johnsonii, Lactobacillus paracasei, Lactobacillus plantarum, Lactobacillus reuteri, Lactobacillus rhamnosus, Lactobacillus lactis.
as bifidobacteria, mention may be made of Bifidobacterium adolescentis, Bifidobacterium bifidum, Bifidobacterium breve, Bifidobacterium infantis, Bifidobacterium lactis (reclassified as Bifidobacterium animalis), Bifidobacterium laterosporus, Bifidobacterium longum, Bifidobacterium thermophilum.
other lactic bacteria notably include Enterococcus faecalis, Enterococcus faecium, Lactococcus lactis, Leuconostoc mesenteroides, Pediococcus acidilactici, Sporolactobacillus inulinus, Streptococcus diacetylactis, Streptococcus intermedius, Streptococcus thermophilus.
as non-lactic bacteria, mention may be made of Bacillus spp, Escherichia coli (Nissle strain), Propionibacterium freudenreichii.

Lactobacilli and bifidobacteria are notably preferred such as the species Bifidobacterium bifidum (bifidus), Bifidobacterium lactis, Bifidobacterium longum, Bifidobacterium breve, Lactobacillus reuteri, Lactobacillus acidophilus, Lactobacillus casei, Lactobacillus plantarum, Lactobacillus rhamnosus.

These micro-organisms with a probiotic purpose are generally commercially available.

Said micro-organisms suitable for the invention are generally isolated, freeze-dried, characterized and/or cultivated before being added and included in the beepollen/clay complex.

In fact, according to a preferred embodiment, this definition does not cover the micro-organisms which are naturally present in the beepollen/clay complex, and which would not have been isolated, characterized, cultivated and/or freeze-dried prior to their inclusion in the complex.

The freeze-dried micro-organisms with a probiotic purpose enter the scope of the present invention.

Micro-organisms with a probiotic purpose are in majority marketed in freeze-dried form. Freeze-drying, or drying under cold condition, is a method based on subdivision which allows the removal of water contained in a material, in a foodstuff or a product in order to make it stable in a room temperature and thereby facilitate its preservation.

Freeze-drying includes many advantages as compared with other drying or preservation methods.
Freeze-drying gives the possibility of preserving a large portion of the qualities of foodstuffs given that the latter remain at a temperature below the freezing point during sublimation. The use of freeze-drying is particularly important in the case of lactic bacteria since they are highly sensitive to heat.
Freeze-dried foodstuffs generally do not require refrigeration for keeping them. Storage and transport cost may thus be reduced in an appreciable way.
Freeze drying causes a significant decrease in the weight, which greatly facilitates transport of freeze-dried foodstuffs. For example, several foodstuffs contain up to 90% of water. They will therefore be ten times lighter after freeze-drying.
Most freeze-dried foodstuffs rehydrate very rapidly by their porous texture. Indeed, freeze-drying does not cause any appreciable decrease in volume. Water may therefore easily regain its position in the molecular structure of the foodstuff.

Freeze-dried micro-organisms are set into a dormant state by drying. They therefore have to be reactivated in order to arrive alive in the intestine and exerting therein a beneficial action for the health of the host, according to the invention.

According to the invention, it has been demonstrated that the beepollen/clay complex represents a support allowing reactivation of these freeze-dried bacteria.

This support therefore represents a favorable environment and ideal for nutrition, protection, development and for freeze-dried micro-organisms, reactivation of the micro-organisms, including when they are administered orally.

Generally, the compositions according to the claim comprise:
from 0.1% to 70% of freeze-dried probiotics; and
from 30% to 99.9% of said complex,

the percentages being understood by weight;

and more particularly:
from 3% to 30% of freeze-dried probiotics; and
from 70% to 97% of said complex,

the percentages being understood by weight.

The bee pollen/clay complexes generally comprise:
from 4% to 90% of beepollen, and
from 10% to 96% of clay

the percentages being understood by weight.

More preferentially, the complexes according to the invention comprise:
from 20% to 40% of beepollen
from 60% to 80% of clay

and more particularly:
from 30% to 35% of beepollen and
from 65% to 70% of clay.

The complexes according to the invention advantageously have a grain size of less than 500 μm, a humidity rate of less than 15% and/or a water activity (aw) of less than 0.75.

Generally, the compositions according to the present invention appear as a powder and notably have the followina characteristics:

| ANALYSES | SPECIFICATIONS |
|---|---|
| ASPECT | Powder |
| COLOR | Dark green-grey to pale green-grey |
| | depending on the amount of |
| | freeze-dried bacteria |
| ODOUR | Specific |
| PH | NA |
| DENSITY | NA |
| HEAVY METALS | Pb < 2 ppm |
| (Pb, As) | As < 1 ppm |

Advantageously, the composition according to the invention may be formulated in a gastro-resistant form, notably in the form of gastro-resistant gelatin capsules.

According to the invention, it was also demonstrated unexpectedly that a gastro-resistant formulation of micro-organisms with a probiotic purpose and of the beepollen/clay complex allows the bioload to be increased with birth intra corpus of ferments while the micro-organisms formulated in the form of gastro-resistant gelatin capsules, without the beepollen/clay complex, had a considerably affected viability level.

Advantageously, when the beepollen/clay complex loaded with freeze-dried probiotics is formulated in gastro-resistant gelatin capsules, the passing at the stomach fits in as a factor favorable to proliferation. Upon contact with the gastric flow contained in the stomach, the gastro-resistant gelatin capsule becomes permeable; water penetrates and provides the humidity which the micro-organisms need for beginning the proliferation phase.

According to the invention, the use of a beepollen/clay complex as a support for orally administering micro-organisms with a probiotic purpose gives the possibility of
protecting the micro-organisms with a probiotic purpose from the deleterious effect of the packaging;
reactivating the freeze-dried micro-organisms with a probiotic purpose included in the composition;
feeding and maintaining viable the micro-organisms with a probiotic purpose included in the composition;
protecting the micro-organisms with a probiotic purpose from gastric acidity;
protecting the micro-organisms with a probiotic purpose from biliary and pancreatic juices;
giving birth intra corpus to micro-organisms with a probiotic purpose adapted to the digestive environment;
releasing the micro-organisms in the intestinal medium while promoting their adhesion and interaction with the intestinal mucosa.

The micro-organisms should bind to the intestine in order to contribute to the reinforcement of the intestinal barrier and exert their beneficial action on the health of the host. Thus, for example, bifidobacteria have capabilities of adhering to the intestinal mucosa where they recognize various receptors of the intestinal epithelium. Lactic bacteria, as for them, occupy potential sites of colonization by pathogens while preventing the penetration of detrimental antigens and thus avoiding the proliferation of pathogenic germs. They consume the available nutrients limiting the source of nutrients for pathogens and competing with the pathogens for accessing the receptors of the hosts. Further, they secrete antimicrobial molecules such as bacteriocins at the epithelium surface and also at the medium (for example acidification).

By increasing the retention of micro-organisms with a probiotic purpose in the intestines, the beepollen/clay complex promotes beneficial interaction of these micro-organisms with the intestinal mucosa. The intestinal microbiota exerts many required physiological functions beneficial for maintaining the health of the host. Maintaining the equilibrium of the intestinal flora and the reinforcement of the intestinal barrier has a major benefit. Micro-organisms with a probiotic purpose are good candidates for achieving this goal. However, major difficulties generally prevent the micro-organisms from exerting a probiotic activity: the requirement of properly feeding the micro-organisms for maintaining them alive; the requirement of reactivating freeze-dried micro-organisms; the low resistance of micro-organisms to shocks of industrialization; the low resistance of micro-organisms to gastric acidity, to biliary and pancreatic juices: the lack of adaptation of the micro-organisms to the digestive environment and the planctonic nature of the micro-organisms which is an obstacle to their adhesion to the mucosa and to their interaction therewith. Strategies for circumventing these difficulties exist but none of them gives the possibility of circumventing the whole of the difficulties. For example, isolated and cultivated beneficial bacteria strains for their resistance to gastric acidity are not reactivated, and the tiny percentage which will reach the intestine will not be able to bind therein or efficiently interact at the intestine. Today, no support is able to simultaneously overcome the various biological and/or physiological obstacles by guaranteeing an action on the target site. The present invention gives the possibility of getting rid to a large extent of these difficulties and provides the micro-organisms with the environment required for their probiotic actions at the intestine.

The bee pollen/clay complex gives the possibility of simultaneously getting rid of the whole of the industrial, biological and/or physiological obstacles to the action of probiotics by providing the following elements:
a nutritive environment required for sustaining the bacteria
a nutritive environment required for reactivating the freeze-dried bacteria
resistance to gastric acidity
resistance to biliary and pancreatic juices
adaptation to the digestive environment
binding to the intestinal wall and delivery

The invention also relates to a method for preparing a composition according to the invention, said method comprises:
the mixing of beepollen and clay;
adding and mixing micro-organisms with a probiotic purpose.

Said micro-organisms with probiotic target may be freeze dried, presented as a powder notably.

Preferably, the obtained mixture may be sifted in order to obtain the desired grain size. Advantageously a sieve with a mesh of 500 μm is used.

The invention also relates to the use of a complex based on beepollen and clay for conveying the micro-organisms with probiotic purposes as far as the intestine, for binding them, improving their bioavailability and/or their probiotic biological activity at the intestine. Said complex is therefore a biovector for administration of micro-organisms with probiotic purposes.

By <<biological activity>> is meant here the nutritional (notably diet effect) and/or physiological effect, such as a probiotic and therapeutic effect exerted by said micro-organisms in subjects which have consumed them.

The present invention therefore notably relates to a food, a cosmetic, a pharmaceutical and/or galenic composition and/or a medical device comprising a composition according to the invention.

Said composition/devices according to the invention are therefore useful for a preventive and/or curative, therapeutic and/or nutritional purpose (notably diet purpose) for human and/or animal use. The excipients required for industrial shaping of the products may be selected from all the acceptable materials according to regulations (foodstuff, food supplement, functional foodstuff, cosmetic, medical, pharmaceutical device), which govern these products.

It is also thus possible to notably mention as excipients the derivatives of cellulose or microcrystalline cellulose, earth alkaline carbonates, magnesium phosphate, starches, modified starches, lactose for solid forms, water, aqueous solutes, saline, isotonic solute, etc.

According to an embodiment, the compositions also comprise a source of sugar such as glucose with which the bacteria may develop.

The composition according to the invention may appear in forms intended for administration via an oral, sublingual, topical, local, intratracheal, intranasal or rectal route, notably an oral route.

They may therefore be notably present as solutes or multidose flasks or as naked or coated tablets, dragees, capsules, soft or hard gelatin capsules, granules, pills, tablets, powders, suppositories or rectal capsules, solutions or suspensions, or further creams, gels, ointments, pastes, patches, food products such as yoghurt, etc.

The present invention is more particularly directed to gastro-resistant formulations such as pastes or gelatin capsules, notably gastro-resistant gelatin capsules comprising a composition according to the invention.

Preferably, said composition contains an effective amount of the compositions according to the invention.

The dose and/or dosage may vary within large limits (0.5 mg to 1,000 mg) and may depend on the administration route as well as on the age and on the weight of the subject.

There may be particular cases when higher or lower dosages are suitable; such dosages do not depart from the scope of the invention. According to customary practice, the dosage suitable for each subject is determined according to the administration method, the weight and the reaction of said subject, as well as according to the concentration of the micro-organisms in said compositions.

The food composition according to the invention may notably be a foodstuff, a functional food (a nutraceutical) or a food supplement.

Functional foodstuffs or nutraceuticals are generally defined as a conventional foodstuff, or which has the aspect thereof, which is part of normal diet, and which has the characteristic of providing beneficial physiological effects exceeding its usual nutritional functions, or of reducing the risks of chronic diseases.

The invention is therefore directed to functional foodstuffs such as yoghurt comprising a composition according to the invention.

Said composition may also form a food supplement in the sense of the European directive 2002/46/EC.

Said food supplement is advantageously formulated as a dose, i.e. in a presentation form as defined by the European Directive 2002/46/EC, such as “gelatin capsules, lozenges, tablets, pills and other similar forms as well as powder sachets, liquid ampoules, flasks provided with a dropper and the other similar forms of liquid or powder preparations intended to be taken in small amount units”.

Said composition may also notably form a pharmaceutical specialty in the sense of the European Directive 2001/83/EC.

Said composition may be useful in preventing and/or treating disorders in which probiotics are generally considered to be beneficial. Mention may thus notably be made of osteoporosis, cardio-vascular diseases, cancers, liver and metabolism disorders, fatigue, circulatory disorders, articular disorders, menopausal symptoms, central nervous system disorders, gastro-intestinal system disorders, eye disorders, urinary disorders, and disorders of the prostate such as benign prostate hypotrophy, chronic prostatitis, prostatodynia and prostate cancer, nutritional, dermatological and cosmeto-dermatological disorders, asthenia and fatigability, convalescence and post-surgery, hydroelectrolytic unbalances, improvements in physical conditions, etc.

The pharmaceutical compositions according to the inventions may also comprise one or several active ingredients.

The present invention also relates to medical devices comprising a composition according to the invention. Said composition may therefore form a medical device in the sense of the European Directive 93/42 EEC.

### FIGURES

FIG. 1 illustrates the development of freeze-dried bacteria (Bifidobacterium longum) in an ideal medium and within the beepollen/clay complex.

FIG. 2 illustrates the gain and loss percentages of ferments after passing into the stomach for four compositions formulated in the form of gastro-resistant gelatin capsules:

i) on a beepollen/clay support;

ii) Bion Transit product,

iii) Lactibiane product ref 2.5G,

iv) Arkoprobiotics defense+

FIG. 3 illustrates the survival of freeze-dried bacteria in gastro-resistant gelatin capsules after passing into the stomach.

FIG. 4 illustrates the reactivation of freeze-dried bacteria associated with the beepollen/clay complex, in gastro-resistant gelatin capsules, after passing into the stomach.

FIG. 5 illustrates the gains and losses of ferments after passing into the stomach and the action of bile and pancreas with or without any complex.

FIG. 6 illustrates the synergistic effect of beepollen and of clay on the gains of ferments in the stomach, chyme, mucosa and intestinal liquid.

The following examples illustrate the invention without however limiting it. The starting products used are known products or prepared according to known operating procedures.

The percentages are expressed by weight, unless indicated otherwise.

### EXAMPLES

Equipment:

Accurate electronic scales—Mettler Toledo PG803

A WTB binder oven

Stirrer—IKA Labotechnik Eurostar

pH-meter from HANNA instruments

glass beaker of 250 mL—Bomex

curved, spoon-shaped spatulas, stainless steel 18/8—Labo-moderne

Suppliers of Raw Materials

Frozen sunflower pollen under nitrogen gas

Pollenergie

La Grabère—47450 St Hilaire de Lusignan

Telephone. 05 53 68 11 11-Fax. 05 53 68 11 12

Montmorillonite clay

Argiletz

14 route d'Echampeau—77 440 Lizy sur Ourcq

Tel. 01 60 61 20 88-Fax. 01 60 61 27 39.

The complex was prepared in the following way.

For preparing 150 g of beepollen/clay complex: the proportion of each of the components is 66.66 of clay for 33.33 percent of beepollen.
Weigh 100 g of clay in a 200 ml beaker;
Weigh 50 g of frozen beepollen in a 250 ml beaker;
The parts of the stirrer are preferably dry for avoiding hydration of the complex;
Mix the clay;
Add the beepollen and let them be mixed for 30 seconds.
The desired grain size is obtained by sifting by means of a sieve with a 500 μm mesh.

The compositions based on beepollen/clay and of micro-organisms with a probiotic purpose are prepared in the following way for 166.67 g of final product with 10% of micro-organisms:
Weigh 100 g of clay in a 200 ml beaker;
Weigh 50 g of frozen beepollen in a 250 ml beaker;
Weigh 16.67 g of micro-organisms in a 200 ml beaker;
The parts of the stirrer are preferably dry for avoiding hydration of the complex;
Mix the clay;
Add the beepollen and let them be mixed for 30 seconds;
Add the micro-organisms and let them mix for 30 seconds;
The desired grain size is obtained by sifting by means of a sieve with a 500 μm mesh

### Example 1: Demonstrating the Beepollen/Clay Complex Capability of Providing a Nutritional Medium and Reactivation of the Freeze-Dried Bacteria

The need for indispensable ingredients for reactivating freeze-dried bacteria was reported in the literature.

These ingredients (sugar, proteins, sodium, etc) and the optimum proportion (needs in %) for reactivating freeze-dried bacteria are shown in the first two columns of the table below. The third column of this table indicates the percentage of these different ingredients contained in the beepollen/clay complex.

| | Needs | In the beepollen/clay |
|---|---|---|
| Ingredients | in % | support in % |
| Peptone (protein hydrolysis) | 16 | 35 |
| Protein | 13 | |
| Yeast | 6.6 | Between 6 and 13 |
| The largest source of vitamin B1 (12 | | |
| mg/100 g) required for metabolism | | |
| of carbohydrates and lipids | | |
| Glucose | 32 | From 20 to 40% |
| Sodium acetate trihydrate | 8 | Sodium provided by the |
| | | clay |
| Ammonium citrate | 3.2 | Provided by the clay |
| Tween (a solubilizer required for | 1.6 | Not applicable |
| assembling non-miscible substances | | |
| together) | | |
| potassium hydrogenphosphate | 3.2 | From 20 to 45% of the |
| | | ashes |
| Magnesium sulfate heptahydrate | 0.3 | From 1 to 12% of the ashes |
| Manganese sulfate tetrahydrate | 0.1 | 1.4% of the ashes * |
| Agar (polysaccharides) | 16 | Between 13 and 55% |
| pH | 6.2 | Pollen at pH 6.0 maintained |
| | | acid by the clay |
| | | Many vitamins, aminoacids |
| | | and growth substances |

A perfect agreement is therefore found between the indispensable elements for reactivating freeze-dried bacteria, as reported in the literature and the elements contained in the beepollen/clay support.

The development of Bifidobacterium longum bacteria within an ideal nutritive medium and within the beepollen/clay complex was demonstrated in the following way.

1—The bacteria were sown in an ideal culture medium and other ones were sown with the beepollen/clay complex in an equivalent amount.

2—Counts were performed for both of these examples.

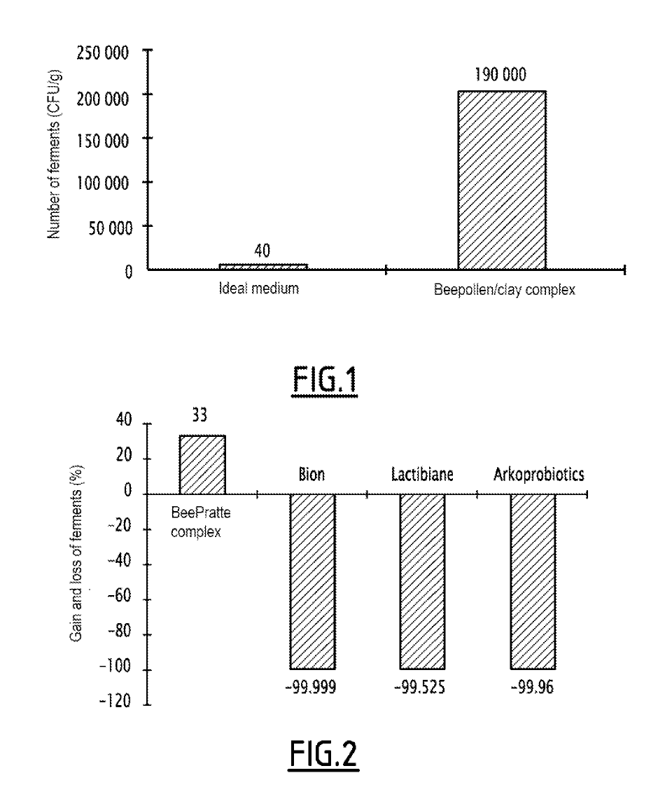

The results illustrated in FIG. 1 show that the beepollen/clay complex allows reactivation and proliferation of the bacteria.

### Example 2: Demonstrating the Resistance to the Acidity of the Beepollen/Clay as Compared with Commercial Product

The three following commercial products were used.

Bion transit

Bion transit provides a specific probiotic strain: Lactobacillus plantarum 299v.

Bion transit is intended for persons for which the intestine is sensitive and which are subject to frequent intestinal disorders: discomfort, difficulties, digestive problems, flatulence, irregular transit, etc.

On the product, a provision of 10,000,000,000 (10 billion) probiotics per gelatin capsule is claimed.

Lactibiane Référence 2.5G

The probiotic strains of Lactibiane Référence are guaranteed to be GRAS (Generally Regarded As Safe) and deposited at the “Collection Nationale de Cultures de Micro-organismes de l'institut Pasteur”. These are the four following strains:
Bifidobacterium longum LA 101
Lactobacillus acidophilus LA 102
Lactococcus Lactis LA 103
Streptococcus thermophilus LA 104

Lactibiane Référence 2.5G contributes to maintaining the intestinal flora, improves digestive comfort and regulates the transit as soon as the first week. It may be an aid in the case of digestive disorders (flatulence, etc) and in preventing winter disorders.

On the product, a provision of 10,000,000,000 (10 billion) of probiotics per gelatin capsule is claimed.

Arkoprobiotics défenses+

Arkoprobiotics défenses+ contains a selection of freeze-dried probiotics stemming from several strains:
Lactobacillus paracasei
Lactobacillus plantarum
Lactobacillus rhamnosus

Arkoprobiotics défense+ is an aid for reinforcing natural defenses of the organism notably during winter and the changes of season, as well as for rebalancing the intestinal flora. It contributes to the equilibrium of the vitality of the organism.

On the product, a provision of 50,000,000,000 (50 billion) of probiotics per gelatin capsule is claimed.

Operating Procedure:

Gastro-resistant gelatin capsules containing the beepollen/clay complex or the bacterial strains of commercial products were manufactured, by means of the kit for preparing gastro-resistant gelatin capsules ApproPharm (254 chemin de la Farlède—83500 La Seyne sur Mer).

The operating procedure for the coating is the following:

Soak the gelatin capsules full of beepollen/clay complex and of commercial products for 10 seconds in the liquid.

Let them dry on a sieve, by frequently stirring in order to avoid any adhesion.

Wait for 10 minutes and then add a second layer: soak the gelatin capsules for 5 seconds in the solution, and then let them dry as earlier.

Wait for 10 minutes and then add a third layer: soak the gelatin capsules for 5 seconds in the solution, and then let them dry as earlier.

Wait for 10 minutes and then add a fourth layer: soak the gelatin capsules for 5 seconds in the solution, and then let them dry as earlier.

Wait for 10 minutes and then add a fifth layer: soak the gelatin capsules for 5 seconds in the solution, and then let them dry as earlier.

Tests were conducted in order to be certain of the resistance of the gelatin capsules in an acid medium. They showed excellent resistance to the acid medium since the capsules were not broken after a period of 4 hours.

Experimental Conditions

The thereby prepared gastro-resistant gelatin capsules were subject to the following experimental conditions.

Thus, the gelatin capsules
were placed in 500 ml (pre-prandial volume of the stomach)
of an acid solution with pH 2 (acidity of the stomach content)
maintained in an oven at a temperature 37° C.(body temperature)
for a total period of 4 hours (duration of the transit at the stomach)
under mechanical stirring (contraction of the stomach)

At the end of the experiment, the gelatin capsules remained intact (no breaking of the gelatin capsule causing dispersion of its contents), but had become slightly permeable. The contents of the gelatin capsules was isolated and analyzed in order to achieve counting of the lactic ferments.

Counting the Lactic Ferments

Counting the lactic ferments was carried out by QSA—Qualité Sécurité Alimentation—Site d'Agropole—BP 125—47931 Agen Cedex 9.

The counting of the lactic ferments present in the beepollen/clay complex within gastro-resistant gelatin capsules and before digestion indicates 60,000 CFU per gram. After digestion, the beepollen/clay complex has a load of lactic bacteria of 80,000 CFU per gram.

For commercial products having been put into gastro-resistant gelatin capsules without any complex, the counting of the lactic ferments after the digestion is the following:

| | | |
|---|---|---|
| Bion Transit | 40 | CFU/g |
| Lactibiane référence 2,5 G | 190,000 | CFU/g |
| Arkoprobiotics défenses+ | 40,000 | CFU/g |

The variations during the digestion of ferments in the beepollen/clay complex and the three commercial products are illustrated in FIG. 2. The commercial products formulated in gastro-resistant gelatin capsules show that less 1% of the ferments are reactivated. The beepollen/clay support as gastro-resistant gelatin capsules gives the possibility of increasing the load of ferments by 33% during digestion.

### Exemple 3: Impact of the Beepollen/Clay Complex on the Reactivation of Freeze-Dried Bacteria

1) Preparation of the Batches
freeze-dried bacteria alone (pure commercial strains)

versus
the same freeze-dried bacteria included in the beepollen/clay complex.

A similar amount of micro-organisms was included in each batch, independently of the amount and/or of the galenic form of the reference product (between 1,000,000 and 2,000,000).

The freeze dried bacteria included in the beepollen/clay complex were prepared in the following way:

The volume of freeze-dried bacteria corresponding to an amount of 1,000,000-2,000,000 was added to the complex. The mixture was homogenized by means of a stirrer for 30 seconds.

2) The gastro-resistant gelatin capsules were prepared according to the procedure of Example 2.

3) The gelatin capsules were then subject to the following experimental conditions. Thus, the gelatin capsules:
were placed in 500 ml (pre-pandial volume of the stomach)
of an acid solution with pH 2 (acidity of the stomach contents)
maintained in an oven having a temperature of 37° C. (body temperature)
for a total period of 4 hours (duration of the transit at the stomach)
under mechanical stirring (contraction of the stomach)

The counting of the lactic ferments was carried out before and after experimentations by an independent microbiological analysis laboratory approved by COFRAC (a French Approval Committee) for program 59—microbiological analysis of agri-feed products, within the scope of approval agreement no. 1344.

The obtained results are the following:
1. Samples without the beepollen/clay complex
Freeze-dried commercial bacteria, before putting them in gelatin capsules, before passing into the stomach (steps 2 and 3 of the procedure not carried out):
Freeze-dried bacteria before passing into the stomach:

| | | |
|---|---|---|
| Bifidobacterium longum | 800 | CFU/g |
| Lactobacillus acidophilus | 41,000,000 | CFU/g |
| Bifidobacterium bifidum | 75,000,000 | CFU/g |

Freeze-dried commercial bacteria, put into gastro-resistant gelatin capsules after passing into the stomach (steps 1, 2 and 3 of the procedure):

| | | |
|---|---|---|
| Bifidobacterium longum | 40 | CFU/g |
| Lactobacillus acidophilus | 160,000 | CFU/g |
| Bifidobacterium bifidum | 32,000 | CFU/g |

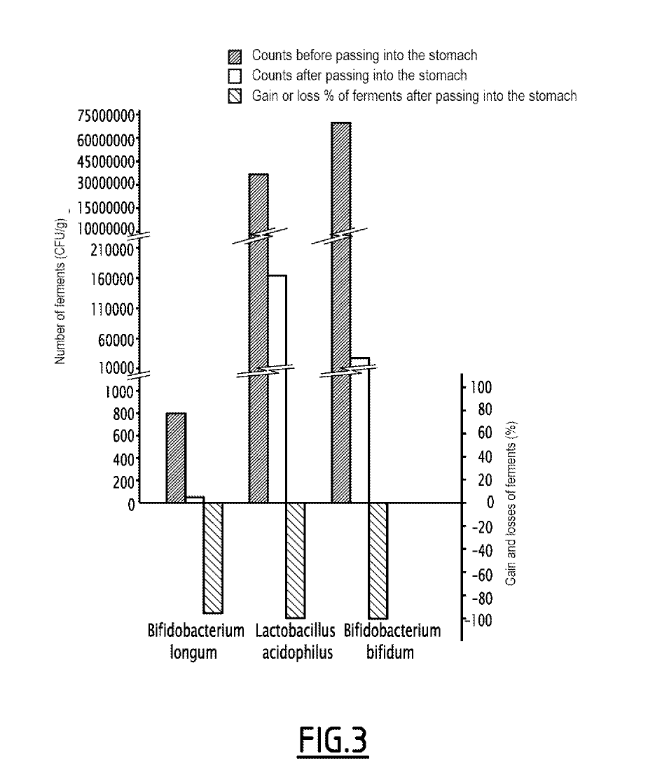

The percentages of gain or loss of lactic ferments after passing into the stomach were calculated. They are illustrated for each bacterium, in FIG. 3.

2. Samples Associated with Beepollen/Clay Complex
Freeze-dried bacteria associated with a complex before putting them in gelatin capsules, before passing into the stomach (steps 2 and 3 of the procedure were not carried out):

| | | |
|---|---|---|
| Bifidobacterium longum | 1,900,000 | CFU/g |
| Lactobacillus acidophilus | 1,800,000 | CFU/g |
| Bifidobacterium bifidum | 1,100,000 | CFU/g |

Freeze-dried bacteria associated with a complex, put into gastro-resistant gelatin capsules after passing into the stomach (steps 2 and 3 of the procedure were not carried out):

| | | |
|---|---|---|
| Bifidobacterium longum | 7,400,000 | CFU/g |
| Lactobacillus acidophilus | 2,600,000 | CFU/g |
| Bifidobacterium bifidum | 2,600,000 | CFU/g |

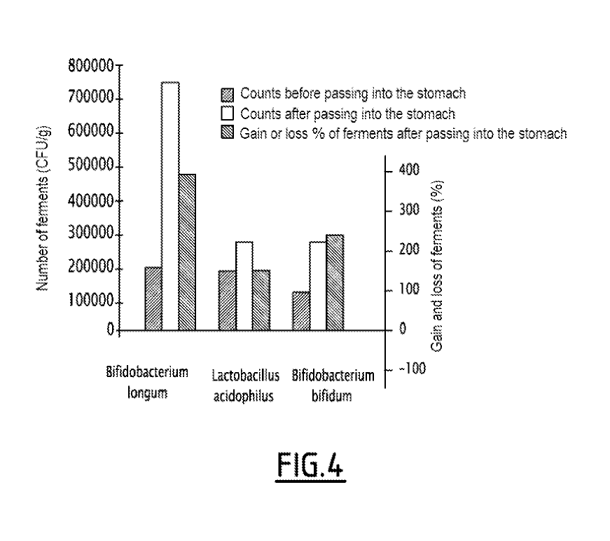

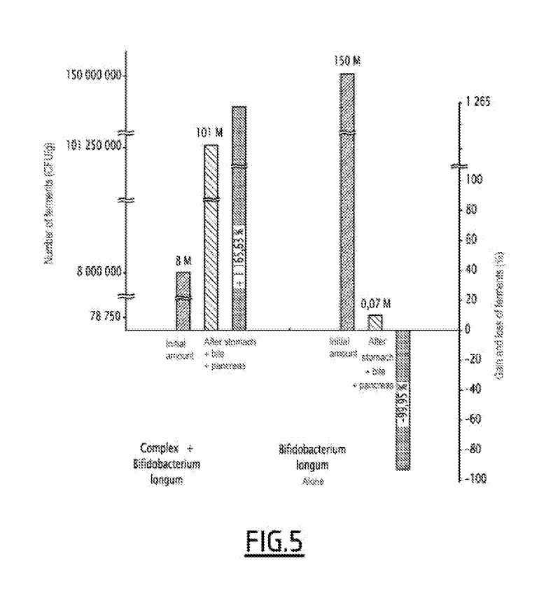

The percentages of gain or loss of the lactic ferments after passing into the stomach were calculated. They are illustrated for each bacterium, either alone or associated with the beepollen/clay complex, in FIG. 4.

The beepollen/clay complex formulated in gastro-resistant gelatin capsules allows an increase in the ferments in the freeze-dried probiotics during digestion.

The freeze-dried probiotics formulated as gastro-resistant gelatin capsules lose at least 95% of their ferments during digestions.

These results show that:
the effect of proliferation of the ferments induced by the beepollen/clay complex on the bacteria naturally included in the fresh beepollen may be extended to the freeze-dried bacteria.
The beepollen/clay complex is a support of choice for reactivating freeze-dried probiotics.
The beepollen/clay complex is used as a support for proliferation, implantation and therefore administration of probiotics.

### Example 4: Intestinal Study

The demonstration that the bacteria which reach the intestine are not degraded by the different secreted juices and that they adhere to the intestinal mucosa is a significant challenge and represents a scientific and industrial potential.

Indeed, when the probiotics reach the intestine, in order to exert their actions on health, they have to resist to the rise of the pH between the stomach and the duodenum, resist to the impact of biliary salts and pancreatic enzymes, in order to adhere to the intestinal wall so as to be able to interact with the support depending on the specificities of the bacteria.

The present study has the purpose of evaluating:
the resistance of probiotics to variations in pH (gastric and intestinal pH) and to the different fluids (biliary salts and pancreatic enzymes)
the adherence of the probiotics to the intestinal wall.

It therefore includes two experiments:
a study on artificial medium giving the possibility of measuring the resistance of the probiotics to physiological variation of pH and to enzymatic affections, from the stomach to the duodenum: this will somewhat complete at the intestinal level the previous studies which studied the resistance of the probiotics to the gastric medium (and for which the results have been reminded earlier);
an ex-vivo study on intestine explants for measuring both the survival of the probiotics in the intestinal lumen and their adhesion to the intestinal mucosa.

### PROCEDURE

### 1. Samples to be Tested

The samples which will have to be tested during this study are:
clay alone
pollen alone
probiotic alone (Bifidobacterium longum)
beepollen/clay complex
beepollen/clay complex+probiotics
Bion, Lactibiane, Arkoprobiotics products

A control (saline) will be produced in parallel, at the same time as these samples.

For homogeneity reasons, all the samples were put into gelatin capsules of size 1, kept at 4° C.

Examples 1-3 having shown that putting into gastro-resistant gelatin capsules of the commercial probiotics like Bifidobacterium longum did not have any benefit as to their survival as compared with the use of conventional gelatin capsules, only the samples including the beepollen/clay complex and the beepollen/clay complex associated with probiotics were packaged into gastro-resistant gelatin capsules according to the procedure hereafter.

Soak the gelatin capsules full of beepollen/clay complex with or without probiotics for 10 seconds in the liquid.

Let them dry on a sieve, by frequently stirring in order to avoid any adhesion.

Wait for 10 minutes and then add a second layer: soak the gelatin capsules for 5 seconds in the solution and then let them dry as earlier.

Wait for 10 minutes and then add a third layer: soak the gelatin capsules for 5 seconds in the solution and then let them dry as earlier.

Wait for 10 minutes and then add a fourth layer: soak the gelatin capsules for 5 seconds in the solution and then let them dry as earlier.

Wait for 10 minutes and then add a fifth layer: soak the gelatin capsules for 5 seconds in the solution and then let them dry as earlier.

2. Simulation In Vitro of the Stomach Phase

The whole of the samples is subject to the procedure of Example 2.
At the stomach, the samples are subject to many physiological parameters which have been reproduced in the following way:
They were diluted in 500 ml (pre-prandial volume of the stomach)
of an acid solution with pH 2 (acidity of the stomach contents)
maintained in an oven temperature of 37° C. (body temperature)
for a total duration of four hours (transit time at the stomach)
with stirring (contraction of the stomach)

After incubation:
in the case of the beepollen/clay complex associated with Bifidobacterium longum, the contents of these gelatin capsules was analyzed from a microbiological point of view;
in the case of non-gastro-resistant gelatin capsules, the stomachal liquid was also analyzed from a microbiological point of view;
62.5 mm of the extract of biliary juices and of pancreatic juice were added to the stomachal liquid. A sample of this liquid (intestinal chyme) was analyzed from a microbiological point of view.

3. Passing from the Gastric pH to the Intestinal pH

At the end of the four hours of incubation, 62.5 ml of the solution consisting of a 0.2 M sodium bicarbonate solution with 1.2% of bile extract and with 0.2% of pancreatic juice were added to the gastric liquid.

4. Studies on Explants: Preparatory Study

4.1. Selecting the Animal Model

The pig was selected as an animal model, this animal having the closest digestive physiology to that of humans: its gastro-intestinal tract is similar to it from the structural point of view (Heinz et al., 1987, J. Gen. Virol. 68, 2495-2499).

4.2. Recovery of the Pig Intestine

The recovered intestines after slaughtering the animals intended for food (without any pathology) are placed in a physiological solution. The solution consists of distilled water and of sodium chloride (NaCl) diluted to 9 per 1,000 (=solution of 0.9% by weight/volume of NaCl, i.e. 9 g/l). It contains 154 mequiv./l of Na⁺ and of Cl⁻ before use, and the intestine is then washed with a Ringer solution.

Longitudinal segments of 6 cm by 27 cm are prepared in the duodeno-jejunum and kept in the Ringer solution.

5. Simulation of the Intestinal Phase

In order to view the adhesion of the microbacteria to the intestinal mucosa, an apparatus was developed, allowing simulation of digestions on explants of pig intestines, with the following parameters:

The content of the gastric chyme adjusted to the proper pH is placed in one of the 700 mm stainless steel receptacles. The contents are continuously stirred so as to simulate the gastric intestinal movements by means of a propeller (20 revolutions per minute)

The pig intestines are placed on plates having the following features:
tilted) (11°) stainless steel plates of 6.8 cm by 22 cm which are covered with a flexible and toothed membrane allowing modeling of peristaltism. This movement will be produced by means of a conveyor belt at the speed of 0.5 cm/minute.

The intestine is then positioned on the plate. When the intestine is properly positioned on the plate, the belt is activated and the flow of the intestinal liquid is launched for a duration of 2 hours, under the following conditions:
maintaining the pig intestine at a constant temperature of 37° C.
Hygrometry is controlled (80%) so as not to dry the pig intestine.
The internal air of the chamber consist of 95% of nitrogen (N₂) and 5% of air.
maintaining the pig intestine in darkness during the experimental phase.

6. Measurements

After 2 hours, the intestines are
removed from their supports;
cut out into transverse sections;
observed under the microscope and scraped in order to produce a microbiological count.

The liquid having passed over the intestine and collected in recovery pans is analyzed from a microbiological point of view.

The liquid before passing over the intestine is also analyzed from a microbiological point of view. On the samples, microbiological analysis of the lactic flora is carried out (NF ISO 15214).

The results relating to the microbiological counts before, during and after passing over the intestine are transmitted as colony-forming-units per gram (CFU). This allows comparison of different levels and evaluation of the possible proliferation. The ratios: analyzed intestine surface areas over the total surface area of the intestine, are taken into account.

Cell morphologies were evaluated. The integrity of the pollen grain is also evaluated.

### RESULTS AND DISCUSSIONS

The goal of this study is to evaluate the resistance of probiotics to gastric acidity, and then to the rise of pH during the transit, to their contacting with biliary salts and pancreatic enzymes, their survival to different intestinal fluids and their adherence to the intestinal wall.

1. Evaluation of the Survival of the Ferments to the Stomachal Phase.

Examples 1-3 shows that digestion does not allow the bacteria to survive if they are not associated with the beepollen/clay complex.

In the same physiological context, the beepollen/clay complex promotes an increase in the amount of ingested probiotics. The bioload increases in the presence of the complex, while, at the same time, the probiotics (Bifidobacterium longum) without the protection of the complex are quasi all killed.

Thus, the passing at the stomach is included as a factor favorable to the proliferation of the bacteria if they are protected by the complex. In contact with water contained in the stomach, the gastro-resistant gelatin capsule becomes permeable, water penetrates and provides the humidity which the bacteria need in order to be reactivated and begin the proliferation phase, provided that the bacteria, as in the case with the complex, have sufficient nutritional reserve.

This crucial reactivation and proliferation step within the complex is the main central line in the probiotic activity of the complex.

### 1. Evaluation of the Survival of the Ferments to Biliary Salts and to Pancreatic Juices.

Gastric acidity and biliary-pancreatic secretions are the main endogenic mechanisms for reactivating the ingested probiotic bacteria.

The complex promotes development intra corpus of new bacteria as soon as the stomachal phase, further improved during the contact with biliary and enzymatic juices. The counts of the flora contained in the intestinal liquid before passing over the intestine give the possibility of determining the contribution of the complex to the survival of the probiotics in contact with the bile and pancreatic juices. Even better than survival, the probiotics continue and enhance their proliferation.

The results obtained show that without any complex, 78,750 probiotics (Bifidobacterium longum) persist at the intestinal liquid. With the complex containing the bacteria, 101,250, 000 are counted. This represents a factor of 46, >100,000,000, 600 respectively as compared with the commercial products tested without the complex (Bion, Lactibiane, Arkoprobiotics, respectively).

Beyond the fact of maintaining them alive, the beepollen/clay complex allows development of lactic bacteria in the intestinal liquid.

Better still: while survival and proliferation in the stomach medium is an essential and determining feature, the phenomenon of resistance and proliferation intra corpus is enhanced upon contacting the bile. An increase by a factor 7.79 between the end of the stomach phase and the beginning of the intestine phase is thus observed.

These results indicate that not only the bacteria are protected by the complex but the latter further ensures their development.

As illustrated in FIG. 5, actually only 0.05% of the initial probiotics remain after gastric passage and the impact of bile and of the pancreatic enzymes if they are not protected. On the other hand, subject to the same conditions, the same probiotics have a gain of 1,165.63% if they are included in the complex.

These results show that the beepollen/clay complex protects the bacteria during digestion both against gastric acidity or pancreatic and biliary juices and promotes their development.

### 3. Evaluation of Probiotics Present in the Intestinal Liquid Collected at the End of Their Flow.

It is also important to observe the load contained in the intestinal liquid which will continue its path along the intestine and the colon.

The obtained results show that the bioload contained in the intestinal liquid collected at the end of the flow is of 77 million ferments with the complex. Without the complex, there are only 3.4 million bacteria in the intestinal liquid i.e. 22.65 times less.

The complex also gives the possibility of having 23.424 and 636 times more bacteria, respectively, than the commercial products (Bion, Lactibiane and Arkoprobiotics respectively).

### 4. Evaluation of the Adhesion of the Complex on the Intestinal Mucosa.

Another determining point of the study relates to the adhesion of the complex and of the bacteria on the intestinal mucosa. In order to have a beneficial action on the mucosa, the probiotics have to adhere thereto.

4.1. Microscopic Evaluation

Photographs were taken before and after the intestinal phase of the study, i.e. before and after the flow of the intestinal liquid which contains the complex with Bifidobacterium longum. A highly significant difference between the mucosa before and after flow is noted. Indeed, an orangey covering is visible over the whole surface of the intestine having been in contact with the intestinal liquid. The photographs show without any ambiguity, a yellow presence characteristic of pollen, this time released from its clay “mantle”.

This visual observation seems to confirm:
the presence of the complex on the intestinal wall in spite of the flow and the associated mechanical force,
the capability of the complex of becoming separate in situ

The observation under the microscope of the intestine gives the possibility of accurately viewing the isolated presence of clay and pollen (either surrounded or not by its clay “mantle”).

With greater magnification, it is possible to view in the mucosa, pollen grains with their clay “mantle”, but also and especially pollen grains cleared of their clayey structure, demonstrating the capability of the pollen grains of getting rid of their clay coating. The grains which have not been released at this stage will be able to be released all along the intestinal path.

These observations indicate that the links between the pollen and the clay may be broken at the intestine and release the ferments and the contents of the pollen. As certain pollen grains are still covered with clay, they may continue their path in the intestine and be gradually disassociated along the digestive tract.

4.2. Microbiological Evaluation

Because of the heterogeneity of a pig intestine, the microbiological data are analyzed with the average of counts of the three added segments to the surface of the investigated intestine.

At the end of the flow of the intestinal liquid over the intestine (end of the digestion), the bioload contained in the intestinal mucus sampled by scraping the mucosa is 582,650 CFU/g for the freeze-dried bacteria (Bifidobacterium longum) versus 5,160,000 as soon as the latter are included in the complex. Therefore, 9 times more bacteria are found at the intestinal mucosa when the beepollen/clay complex is used as a vector. The complex gives the possibility of having 69, 301 and 818 times more bacteria respectively at the intestinal mucosa than the commercial products (Lactibiane, Bion and Arkoprobiotics respectively).

In the liquid harvested after passing over the intestine, the ratio between the bacteria present in the complex and the bacteria without any complex was more than 22 times. This difference between both of these ratios is explained by the fact that the very significant bioload of the intestinal liquid during the use of the complex as a support for probiotics has saturated the intestinal mucosa with microferments.

Further, this allows the bacteria contained in the intestinal liquid (and which will continue to multiply subsequently along the intestinal path) to colonize the following portions of the digestive tract.

The beepollen/clay complex is therefore a good factor promoting adhesion of probiotics to the intestinal mucosa.

4.3. Evaluation of the Imprints

Lactic flora has better adherence when it is associated with the complex. Peripheral action is also observed on the total flora. In addition to the benefits of the complex for lactic flora, it seems to act on the control of the total flora, the latter being significantly less substantial in the presence of the complex.

The development of the total flora is totally different on the imprints of the intestinal segment without or with a complex. The Bifidobacterium longum bacteria alone generate imprints with highly significant development of the total flora (innumerable on the imprint). On the other hand, with the complex associated with the Bifidobacterium longum bacteria, very few colonies (between 10 and 20) develop. Oppositely, a significant development of lactic flora is observed. This means that the flora which is desirably implanted (the probiotics) will actually compete with the total flora at the digestive mucosa where it is implanted to the detriment of the latter: this is exactly what is desired during the use of probiotics.

The global conclusions of the results mentioned in paragraphs 2 to 4 of this part are therefore the following:
the complex allows survival of the probiotics to the attack from biliary and pancreatic juices;
better, it allows highly significant multiplication of these probiotics, the latter being adapted to this medium;
the complex allows adhesion of these probiotics to the intestinal mucosa;
the pollen may gradually be detached from the complex in order to release the bacteria;
the very large amount of the provided flora and its great viability allow it to be implanted in the local flora.

### 5. Evaluation of the Synergy of the Ingredients of the Beepollen/Clay Complex

Among the samples used in this study, one included clay alone and another one pollen alone. It is therefore possible to estimate the benefit of the complex by comparing the expected effect by calculating, with weighting, the effect of the constituents taken individually and the results obtained by the study: the synergistic effect of the complex is observed:

| | STOMACH | | CHYME | | MUCOSA | |
|---|---|---|---|---|---|---|
| | % | UFC/g | % | CFU/g | % | CFU/g |
| Bifidobacterium | 0.00% | 0 | 0.08% | 78,750 | 11.29% | 582,65 [symbol] |
| longum (BL) | | | | | | |
| clay | 0.00% | 0 | 0.03% | 30,375 | 3.38% | 174,58 [symbol] |
| Pollen | 0.00% | 0 | 0.00% | 0 | 2.29% | 118,25 [symbol] |
| Complex + BL | 0.00% | 0 | 0.11% | 109,125 | 16.97% | 875,48 [symbol] |
| (estimated total) | | | | | | |
| Complex + BL | 100% | 13,000,000 | 100% | 101,250,000 | 100% | 5,160,00 [symbol] |
| (results) | | | | | | |
| Synergistic effect | | | 92.600% | ×927.8 | +489% | ×5. [symbol] |

[symbol]

indicates data missing or illegible when filed

It is therefore possible to thereby view the synergistic effect at all the stages of the complex:

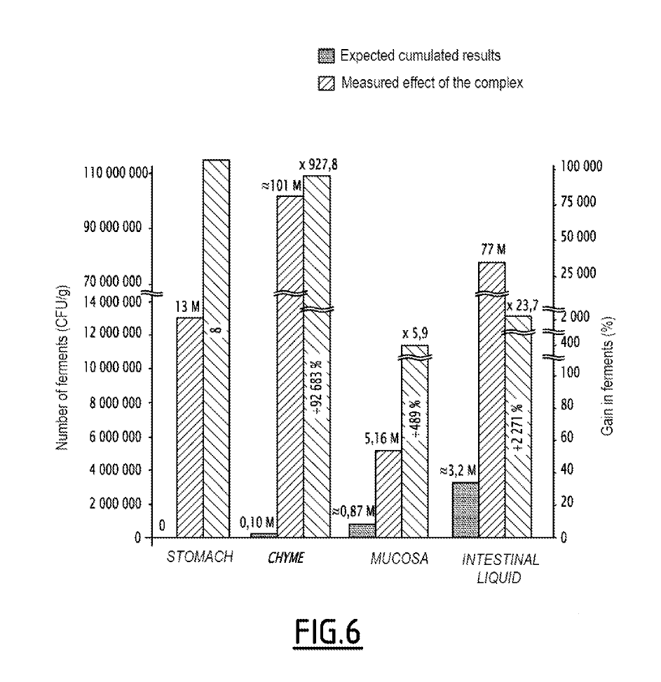

The synergistic effect of the beepollen and of the clay on the gains in ferments in the stomach, chyme, mucosa, intestinal liquid is illustrated in FIG. 6.

The complex allows an increase in the number of lactic bacteria by 92,683% in the chyme, 489% at the intestinal mucosa and 2,271% in the intestinal liquid, as compared with the estimated values.

These ex-vivo results obtained on pig intestines allowed evaluation of the resistance of the probiotics to gastric acidity and to the rise in pH during the transit, to the contacting with biliary salts and pancreatic juices and their survival to different intestinal fluids as well as their adherence to the intestinal wall.

The beneficial contribution of the beepollen/clay complex was demonstrated on all the phases of digestion.

### CONCLUSIONS

During the stomachal phase, when it is packaged in gastro-resistant gelatin capsules, the beepollen/clay complex allows the ferments which are associated with them to be reactivated. This is a supportive choice for reactivation and proliferation of “new” lactic bacteria.

During the intestinal phase, the complex is in a first phase put into contact with bile and the pancreatic juices without this affecting the cell viability since on the contrary, the proliferation capacity remains and increases.

During the intestinal transit, the complex easily adheres to the mucosa. Microscopic observations show that the pollen grains are released from their clay “mantle”, releasing in situ their cytoplasm. The count of ferments on the intestinal mucosa gives the possibility of stating that the complex actually releases its bioload at the intestine both on the intestinal wall and the flow of the intestinal lumen. The major benefit differentiating the complex consists in the synergistic action of certain intrinsic features of the components such as the absorbing effect of clay. The nutritive contribution of the pollen is thus completed by its capability of opposing irreversible adsorption of the bacteria at the clay. This increase in probiotics on the surface of the intestinal mucosa also plays a role on the total flora, the development of which is reduced.

1. A composition comprising:
one or several micro-organisms with a probiotic purpose, added to
a complex based on beepollen and clay.

2. The composition according to claim 1, such that said micro-organisms are freeze-dried.

3. The composition according to claim 1, such that said micro-organisms are selected from lactic bacteria.

4. The composition according to claim 1,such that said micro-organisms are selected from bacteria of the Bifidobacterium spp and Lactobacillus spp genera.

5. The compositions according to claim 1, comprising,
from 0.1% to 70% of freeze dried micro-organisms; and
from 30% to 99.9% of said complex,
the percentages being understood by weight.

6. The composition according to claim 1, comprising:
from 3% to 30% of freeze-dried micro-organisms; and
from 70% to 97% of said complex,
the percentages being understood by weight.

7. The compositions according to claim 1, such that said complex comprises:
from 20% to 40% of beepollen and
from 60% to 80% of clay.

8. The composition according to claim 1 such that it appears as a powder.

9. The composition according to claim 1 in a gastro-resistant formulation.

10. The composition according to claim 9, formulated as gastro-resistant gelatin capsules.

11. A method for preparing a composition according to claim 1 comprising:
mixing beepollen and clay; and
adding and mixing micro-organisms.

12. A food, cosmetic, and/or galenic composition comprising a composition according to claim 1.

13. The food composition according to claim 12, such that it is a foodstuff, a functional foodstuff or a food supplement.

14. A pharmaceutical composition comprising a composition according to claim 1.

15. The use of a complex based on beepollen and clay for improving bioavailability and/or activity of micro-organisms with a probiotic purpose.

16. The pharmaceutical compositions according to claim 14 for its use in order to improve bioavailability and/or activity of micro organisms with a probiotic purpose.

17. A method of treatment, comprising administering a micro-organism with a probiotic purpose and a pharmaceutical composition according to claim 14.
